(19) United States
(12) Patent Application Publication
(10) Pub. No.: US 2022/0093274 A1
(43) Pub. Date: Mar. 24, 2022

(54) INTEGRATED CONTACT TRACING PLATFORM

(71) Applicant: DAV ACQUISITION CORP., Orlando, FL (US)

(72) Inventors: Jennifer SETHRE, Austin, TX (US); Aquiles MATA, Lakeway, TX (US); Adolfo SALUME, San Salvador (SV); Michael A. LIBERTY, Windermere, FL (US)

(73) Assignee: DAV ACQUISITION CORP., Orlando, FL (US)

(21) Appl. No.: 17/029,972

(22) Filed: Sep. 23, 2020

(51) Int. Cl. G16H 50/80; G16H 10/60; G16H 50/70; G16H 50/20; G16H 10/40; G16H 10/20; G06F 16/23; G06N 20/00

(52) U.S. Cl. CPC G16H 50/80; G16H 10/60; G16H 50/70; G16H 50/20; A61B 5/7282; G16H 10/20; G06F 16/2358; G06N 20/00; G16H 10/40

(57) ABSTRACT

A contact tracing protocol performed by an integrated contact tracing platform is disclosed. For example, a method comprises: receiving location information associated with a first participant in the contact tracing protocol; receiving medical information associated with the first participant, the medical information indicating the first participant has contracted an infectious medical condition; identifying, based on the location information, a plurality of participants in the contact tracing protocol who came into contact with the first participant during a contagious period associated with the infectious medical condition; receiving medical information associated with a second participant of the plurality of participants, the medical information related to the infectious medical condition; determining, based on the medical information associated with the second participant, a likelihood that the second participant has contracted the infectious medical condition; and generating a notification for each participant of the plurality of participants indicating an exposure to the infectious medical condition.

### BACKGROUND

Contact tracing can be used to control communicable disease. Contact tracing is the process of identifying persons, or “contacts,” who may have come into contact with a person infected with a communicable disease and subsequently collecting information about the contacts. By tracing the contacts of an infected person, the contacts can be tested for infection, quarantined, and/or treated if infected. This process can reduce the spread of the infection within the population. Non-limiting examples of communicable diseases for which contact tracing is commonly performed for include: tuberculosis, vaccine-preventable infections (such as measles), sexually transmitted infections (such as HIV), blood-borne infections, virus diseases (such as Ebola), bacterial infections, and novel infections, including SARS-CoV, H1N1, and COVID-19.

### SUMMARY

Representative embodiments set forth herein disclose various techniques for enabling a system and a method for contact tracing.

In one embodiment, a computer-implemented method for a contact tracing protocol performed by an integrated contact tracing platform is disclosed. The method comprises: receiving location information associated with a first participant in the contact tracing protocol; receiving medical information associated with the first participant, the medical information indicating the first participant has contracted an infectious medical condition; identifying, based on the location information, a plurality of participants in the contact tracing protocol who came into contact with the first participant during a contagious period associated with the infectious medical condition; receiving medical information associated with a second participant of the plurality of participants, the medical information related to the infectious medical condition; determining, based on the medical information associated with the second participant, a likelihood that the second participant has contracted the infectious medical condition from the first participant; and generating a notification for each participant of the plurality of participants indicating a potential exposure to the infectious medical condition.

In another embodiment, a system, comprises: a memory storing instructions that implement an application for reconciling electronic health records of a patient; and a processing device communicatively coupled to the memory. The processing device is capable of executing the application to: receive location information associated with a first participant in the contact tracing protocol; receive medical information associated with the first participant, the medical information indicating the first participant has contracted an infectious medical condition; identify, based on the location information, a plurality of participants in the contact tracing protocol who came into contact with the first participant during a contagious period associated with the infectious medical condition; receive medical information associated with a second participant of the plurality of participants, the medical information related to the infectious medical condition; determine, based on the medical information associated with the second participant, a likelihood that the second participant has contracted the infectious medical condition from the first participant; and generate a notification for each participant of the plurality of participants indicating a potential exposure to the infectious medical condition.

In still yet another embodiment, a tangible, non-transitory computer-readable medium storing instructions that, when executed, cause a processing device to: receive location information associated with a first participant in the contact tracing protocol; receive medical information associated with the first participant, the medical information indicating the first participant has contracted an infectious medical condition; identify, based on the location information, a plurality of participants in the contact tracing protocol who came into contact with the first participant during a contagious period associated with the infectious medical condition; receive medical information associated with a second participant of the plurality of participants, the medical information related to the infectious medical condition; determine, based on the medical information associated with the second participant, a likelihood that the second participant has contracted the infectious medical condition from the first participant; and generate a notification for each participant of the plurality of participants indicating a potential exposure to the infectious medical condition.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 generally illustrates a block diagram of an example system for performing a contact tracing protocol according to the principles of the present disclosure.

FIGS. 2-5 generally depict methods for performing the contact tracing protocol according to the principles of the present disclosure.

FIG. 6 generally illustrates a detailed view of a computing device that can be used to implement the various components described herein according to the principles of the present disclosure.

### NOTATION AND NOMENCLATURE

Various terms are used to refer to particular system components. Different companies may refer to a component by different names—this document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection or through an indirect connection via other devices and connections.

The terminology used herein is for the purpose of describing particular example embodiments only, and is not intended to be limiting. As used herein, the singular forms “a,” “an,” and “the” may be intended to include the plural forms as well, unless the context clearly indicates otherwise. The method steps, processes, and operations described herein are not to be construed as necessarily requiring their performance in the particular order discussed or illustrated, unless specifically identified as an order of performance. It is also to be understood that additional or alternative steps may be employed.

The terms first, second, third, etc. may be used herein to describe various elements, components, regions, layers and/or sections; however, these elements, components, regions, layers and/or sections should not be limited by these terms. These terms may be only used to distinguish one element, component, region, layer, or section from another region, layer, or section. Terms such as “first,” “second,” and other numerical terms, when used herein, do not imply a sequence or order unless clearly indicated by the context. Thus, a first element, component, region, layer, or section discussed below could be termed a second element, component, region, layer, or section without departing from the teachings of the example embodiments. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C. In another example, the phrase “one or more” when used with a list of items means there may be one item or any suitable number of items exceeding one.

Some embodiments are described in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, or the like.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment.

Contact tracing can be used to control communicable disease. Contact tracing is the process of identifying persons, or “contacts,” who may have come into contact with a person infected with a communicable disease and subsequently collecting information about the contacts. By tracing the contacts of an infected person, the contacts can be tested for infection, quarantined, and/or treated if infected. This process can reduce the spread of the infection within the population. Non-limiting examples of communicable diseases for which contact tracing is commonly performed for include: tuberculosis, vaccine-preventable infections (such as measles), sexually transmitted infections (such as HIV), blood-borne infections, virus diseases (such as Ebola), bacterial infections, and novel infections, including SARS-CoV, H1N1, and COVID-19. Non-limiting examples of benefits of contact tracing may include: interrupting ongoing transmission and reducing the spread of an infection, notifying contacts of the possibility of infection and preventive services or prophylactic care, providing infected contacts counseling and treatment, and learning about the epidemiology of a communicable disease within a particular population.

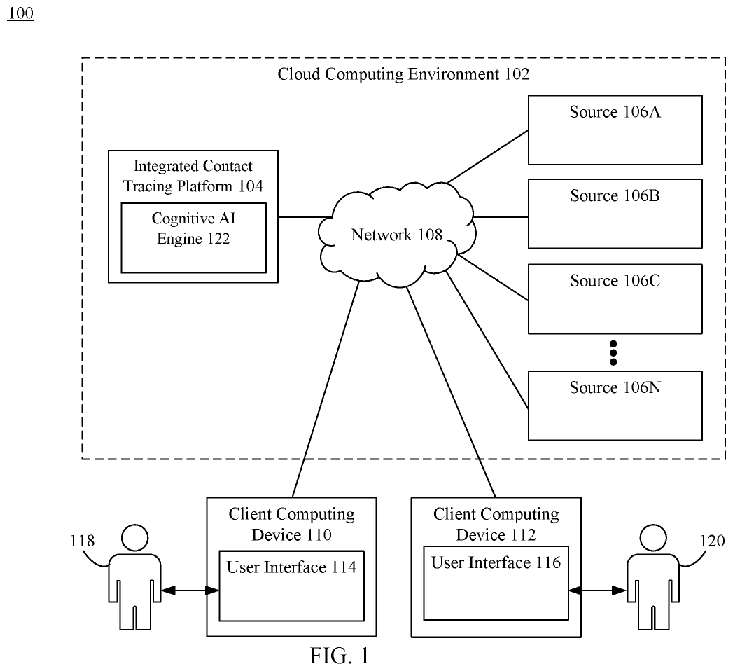

To help illustrate, FIG. 1 will now be described. FIG. 1 generally depicts a block diagram of an example system 100 for performing a contact tracing protocol, according to an embodiment. In FIG. 1, system 100 comprises a cloud computing environment 102 and a plurality of computing devices, such as a client computing device 110 and a client computing device 112. As further shown in FIG. 1, an integrated contact tracing platform 104 can be communicatively coupled, via a network 108, to computing device 110 and computing device 112.

Network 108 may comprise one or more networks such as local area networks (LANs), wide area networks (WANs), enterprise networks, the Internet, etc., and may include one or more of wired and/or wireless portions. Computing device 110 and computing device 112 may include at least one network interface that enables communications over network 108. Examples of such a network interface, wired or wireless, include an IEEE 802.11 wireless LAN (WLAN) wireless interface, a Worldwide Interoperability for Microwave Access (Wi-MAX) interface, an Ethernet interface, a Universal Serial Bus (USB) interface, a cellular network interface, a Bluetooth™ interface, a near field communication (NFC) interface, etc.

Client computing devices 110 and 112 may be any type of stationary or mobile computing device, including a mobile computer or mobile computing device (e.g., a smart phone, a laptop computer, a notebook computer, a tablet computer such as an Apple iPad™ a netbook, etc.), a wearable computing device (e.g., a smart watch, a head-mounted device including smart glasses such as Google® Glass™, etc.), a stationary computing device such as a desktop computer or PC (personal computer), any combination thereof, or any other desired computing device. Client computing devices 110 and 112 may each be associated with a user or a “participant” in the contact tracing protocol. As shown in FIG. 1, client computing devices 110 and 112 can be associated with a participant 118 and a participant 120, respectively.

As further illustrated in FIG. 1, a cloud computing environment 102 can include an integrated contact tracing platform 104 and sources 106A, 106B, 106C . . . 106N. Integrated contact tracing platform 104 can be communicatively coupled via network 108 to sources 106A, 106B, 106C . . . 106N. In one embodiment, integrated contact tracing platform 104 may be a cloud service/application running in one or more resources in cloud computing environment 102. Sources 106A, 106B, 106C . . . 106N may include websites hosted on or cloud services/applications running in one or more resources in cloud computing environment 102. For example, the one or more resources may include one or more servers and form a network-accessible server set that are each accessible by a network such as the Internet (e.g., in a “cloud-based” embodiment) to store, manage, and process data. Additionally, cloud computing environment 102 may include any type and number of other resources including resources that facilitate communications with and between the servers (e.g., network switches, networks, etc.), storage by the servers (e.g., storage devices, etc.), resources that manage other resources (e.g., hypervisors that manage virtual machines to present a virtual operating platform for tenants of a multi-tenant cloud, etc.), and/or further types of resources. As such, integrated contact tracing platform 104 may be implemented in various ways.

As portrayed in FIG. 1, client computing devices 110 and 112 include user interface 114 and user interface 116, respectively. In an embodiment, user interface 114 and user interface 116 may be example components of a cloud application hosted in cloud computing environment 102, where user interface 114 and user interface 116 are front-end components of the cloud application and integrated contact tracing platform 104 is a back-end component of the cloud application. For example, user interface 114 and user interface 116 may be represented as a web page displayed in a web browser executing on client computing devices 110 and 112, respectively. As another example, user interfaces 114 and 116 may be Internet-enabled applications executing on client computing device 110 and client computing device 112, respectively. As such, in accordance with embodiments described herein, a user interface (such as user interface 114 and user interface 116) of integrated contact tracing platform 104 may be implemented in one or more end user computing devices (such as client computing devices 1106 and 112), and integrated contact tracing platform 104 may be implemented on one or more servers that are accessible to the one or more end user computing devices via one or more networks (such as network 108). Still other implementations of user interface 114 and user interface 116 are possible.

Integrated contact tracing platform 104 is configured to receive location information associated with participants in the contact tracing protocol. For example, integrated contact tracing platform 104 may receive location information associated with participants 118 and 120 from client computing devices 110 and 112, respectively, via network 108. The location information may indicate location of participant 118 at a particular time. Integrated contact tracing platform 104 is further configured to perform the contact tracing protocol based on the location information. For example, client computing devices 110 and 112 may provide the location information useful for performing principles of the contact tracing protocol using GPS, Bluetooth, and/or WiFi signals.

In addition, integrated contact tracing platform 104 can be configured to receive medical information associated with participants in the contact tracing protocol. For example, integrated contact tracing platform 104 may receive medical information associated with participants 118 and 120 from sources 106A, 106B, 106C . . . 106N, via network 108. More specifically, integrated contact tracing platform 104 can be configured to receive medical information related data maintained by sources 106A, 106B, 106C . . . 106N. Sources 106A, 106B, 106C . . . 106N may be associated with different entities. For example, any of the entities may be part of a healthcare ecosystem and include medical personnel (e.g., physicians, nurses, pharmacists, dentists, optometrists, orthodontists, etc.), insurance providers, clinics, hospitals, pharmacies, professional associations, government agencies, health information exchanges (HIE), e-prescribing solution providers, and/or the like. In an embodiment, integrated contact tracing platform 104 is also configured to receive medical information from client computing devices 110 and 112. For example, participants 118 and 120 of client computing devices 110 and 112 may interact with integrated contact tracing platform 104 through user interfaces 114 and 116, respectively. As another example, a user (such as a health provider professional including doctors, nurses, physical therapists, pharmacist, etc.) may interact with integrated contact tracing platform 104 through a user interface executing on a client computing device. In an embodiment, participant 118 may request through integrated contact tracing platform 104 his or her medical information maintained by a source (e.g., sources 106A, 106B, 106C . . . 106N) via user interface 114.

FIG. 1 is not intended to be limiting: system 100 may include more or fewer components than those illustrated in FIG. 1.

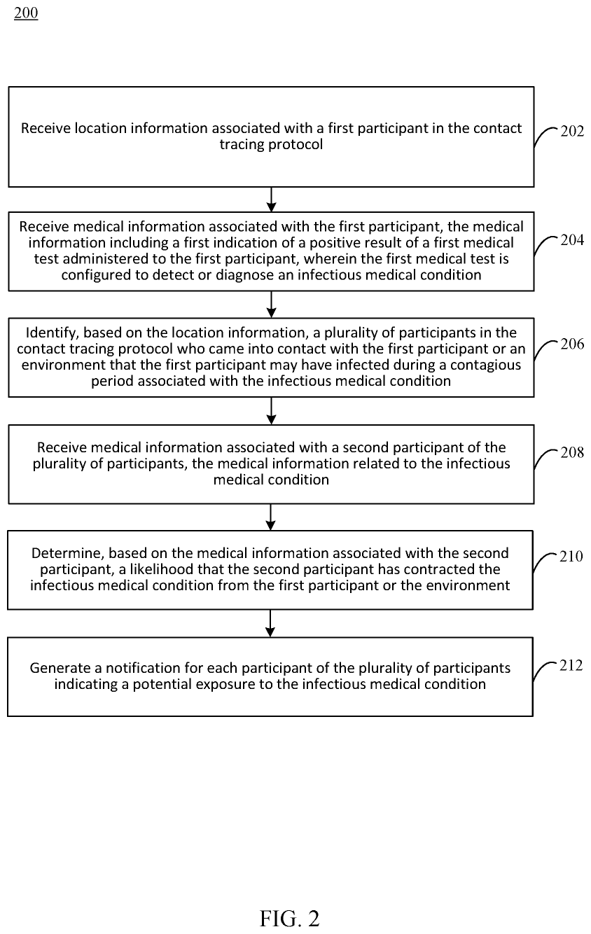

Integrated contact tracing platform 104 is further configured to perform the contact tracing protocol based on the medical information. To explore this in further detail, FIG. 2 will now be described. FIG. 2 generally illustrates a method 200 for performing a contact tracing protocol. As shown in FIG. 2, method 200 starts at step 202.

At step 202, location information associated with a first participant in the contact tracing protocol is received. For example, with continued reference to FIG. 1, integrated contact tracing platform 104 may receive location information associated with the first participant, such as participant 118, from client computing device 110 via network 108. In an embodiment, participant 118 may consent, via user interface 114, to integrated contact tracing platform 104 receiving location information associated with participant 118 from computing device 110. Client computing devices 110 and 112 may provide the location information useful for performing the contact tracing protocol using GPS, Bluetooth or WiFi signals. For example, the location information may include a route that a participant traveled, buildings in which the participant was located within, an address or GPS coordinates in which a person was present, any other location information or combination thereof. The location information may include a date indicator and/or a time indicator associated with the location information. For example, the date indicator and time indicator may include the following date and time indicators for the participant on a particular day: 1) Date: Aug. 6, 2020, Time: 12:00 am-8:25 am, Location: Home; 2) Date: Aug. 6, 2020, Time: 8:25 am-8:35 am, Location: route traveled to ABC Grocery Store; 3) Date: Aug. 6, 2020, Time: 8:35 am-9:30 am, Location: ABC Grocery Store; 4) Date: Aug. 6, 2020, Time: 9:30 am-9:40 am, Location: route traveled to Home; and 5) Date: Aug. 6, 2020, Time: 9:40-11:59 pm, Location: Home. In an embodiment, participant 118 may self-report his or her position. For example, participant 118 may report his or her location through a “check-in” via user interface 114.

At step 204, medical information associated with the first participant is received, where the medical information indicates that the first participant has contracted an infectious medical condition. Medical condition, as used herein, may refer to a biological or psychological state which is not within the range of normal human variation, such as a disease, illness, injury, any physiologic, mental or psychological condition or disorder, etc. For example, and with continued reference to FIG. 1, integrated contact tracing platform 104 may receive medical information associated with participant 118 (e.g., from sources 106A, 106B, 106C . . . 106N). For illustration purposes, a laboratory or a healthcare professional may administer a medical test that is performed to detect or diagnose the infectious medical condition to participant 118. In accordance with this example, the medical information provided to integrated contact tracing platform 104 from the laboratory or the healthcare professional of participant 118 may indicate a positive result that the participant has contracted an infectious medical condition. As another example, integrated contact tracing platform 104 may receive medical information associated with participant 118 from client computing device 110. In accordance with this example, participant 118 may indicate, via user interface 114, that he or she has contracted the infectious medical condition.

At step 206, a plurality of participants in the contact tracing protocol are identified based on the location information, where the plurality of participants came into contact with the first participant during a contagious period associated with the infectious medical condition. Contact as used herein refers to physical and non-physical encounters between participants. For example, depending upon the infectious medical condition, two participants encountering each other within a certain distance (e.g., within six feet of each other) may be considered a contact. For example, a contagious period for COVID-19 may be a 14 day period in which an infected person is contagious to others.

To help further illustrate step 206, integrated contact tracing platform 104 may identify the plurality of participants who came into contact with participant 118 during the contagious period. The identification may be performed by comparing the location information of participants received by integrated contact tracing platform 104 to the location information associated with the first participant collected during the contagious period. The plurality of participants may have location information that indicates that the plurality of participants were at a same location as participant 118 at a substantial similar time and/or at a time subsequent to participant 118 being at that location. For example, the location information may reveal that participant 118 and the plurality of participants attended the same concert during the contagious period of participant 118. As another example, the location information may reveal that the time spent by participant 118 and the plurality of participants at a restaurant overlapped.

In some embodiments, integrated contact tracing platform 104 may identify, based on the location information, a plurality of participants in the contact tracing protocol who came into contact with an environment or surface that participant 118 may have contaminated with the infectious medical condition during the contagious period associated with participant's infectious medical condition. For example, depending upon the how the infectious medical condition is spread between people, integrated contact tracing platform 104 may identify participants who may not have had contact with participant 118 but may have handled objects or were in an environment that participant 118 may have contaminated. For illustration purposes, if a particular pathogen lingers on a surface for a period of time, any participant may be identified who may have come into contact with the surface.

Similarly, if a particular pathogen lingers in the air of an environment for a period of time, any participant who may have been in the environment within this period may be identified.

At step 208, medical information associated with a second participant of the plurality of participants is received, where the medical information is related to the infectious medical condition. For example, and with continued reference to FIG. 1, integrated contact tracing platform 104 may receive medical information associated with a second participant, such as participant 120 (e.g., from sources 106A, 106B, 106C . . . 106N). For illustration purposes, a laboratory or a healthcare provider may administer a medical test for detecting or diagnosing the infectious medical condition of participant 120. In accordance with this example, the medical information provided to integrated contact tracing platform 104 may include a negative result indicating that participant 120 has not contracted the infectious medical condition. For example, the medical information associated with participant 120 may include an indication of a negative result of a medical test administered to participant 120 (e.g., testing a biological sample taken from participant 120). The medical test can be configured to detect or aid in diagnosis of the infectious medical condition. In this case, the medical test is administered after a contact between participant 118 and participant 120 occurs but does not detect the infectious medical condition. As another example, the medical information associated with participant 120 may include indications of symptoms associated with the infectious medical condition that are experienced by the second participant. In the case of COVID-19, symptoms may include fever or chills, cough, shortness of breath or difficulty breathing, fatigue, muscle or body aches, headache, new loss of taste or smell, sore throat, congestion or runny nose, nausea or vomiting, or diarrhea.

At step 210, based on the medical information associated with the second participant, a likelihood that the second participant has contracted the infectious medical condition from the first participant is determined. For example, and with continued reference to FIG. 1, integrated contact tracing platform 104 may analyze the medical information associated with participant 120 to determine a likelihood that second participant 120 has contracted the infectious medical condition from participant 118. For example, the medical information may include results of a medical test, a record of an appointment scheduled for participant 120 to take the medical test, a record of an appointment scheduled for participant 120 to meet with a healthcare provider following the administration of the medical test, or notes of the healthcare provider discussing results of the medical test. A healthcare provider may refer to a doctor, physician assistant, nurse, lab technician, or the like. A healthcare provider may refer to any person with a credential, license, degree, or the like in the field of medicine. In an embodiment, the medical information may indicate that participant 120 received the medical test and the notes of the doctor may confirm the results of the medical test reported by a laboratory. In an embodiment, notes from a healthcare provider may reveal symptoms participant 120 was experiencing at or before the time of the appointment. If the symptoms do not match symptoms associated with the infectious medical condition, then integrated contact tracing platform 104 may determine that second participant 120 has a low likelihood of having contracted the infectious medical condition. In an embodiment, the medical information may indicate that participant 120 has tested positive for antibodies for an infectious medical condition. Integrated contact tracing platform 104 may determine that second participant 120 has an immunity to the infectious medical condition and has not contracted the infectious medical condition.

At step 212, a notification is generated for each participant of the plurality of participants, where the notification indicates a potential exposure to the infectious medical condition. For example, integrated contact tracing platform 104 may generate the notification for each participant of the plurality of participants. Integrated contact tracing platform 104 may provide the notifications to each participant of the plurality of participants via one or more user interfaces executing on one or more computing devices associated with each participant of the plurality of participants. For example, in an embodiment, notifications may be provided to each of the plurality of participants via text messages or a phone call. For example, integrated contact tracing platform 104 may generate a notification for participant 120 and provide the notification to computing device 112 to be displayed on user interface 116. Similarly, each of the plurality of participants may have a computing device (e.g., the same and/or comparable to computing device 112) configured to receive the notification from integrated contact tracing platform 104 and display the notification on a user interface (e.g., the same and/or comparable to user interface 116). In another embodiment, the medical information associated with participant 120 may include an indication of a negative result of a medical test administered to participant 120. As such, when generating the notification for each participant of the plurality of participants, integrated contact tracing platform 104 may exclude participant 120 as it has been determined that participant 120 has not contracted the infectious medical condition.

As further shown in FIG. 1, integrated contact tracing platform 104 includes cognitive artificial intelligence (AI) engine 122. Cognitive AI engine 122 may include a machine learning (ML) model generator and ML models. The ML model generator may be configured to generate ML models to facilitate the determination of a likelihood that a participant has contracted the infectious medical condition by cognitive AI engine 122. Further, the ML models may be deployed in cognitive AI engine 122. Cognitive AI engine 122 is further configured to receive medical information from sources 106A, 106B, 106C . . . 106N in FIG. 1 and/or receive medical information from client computing device 110 and client computing device 112.

Further, cognitive AI engine 122 may use natural language processing (NLP), data mining, and pattern recognition technologies to process and analyze the medical information. More specifically, cognitive AI engine 122 may use different AI technologies to understand language, translate content between languages, recognize elements in images and speech, and perform sentiment analysis. For example, cognitive AI engine 122 may rely on NLP technologies for the recognition and translation of spoken language in content and for understanding of natural language in written content. As another example, cognitive AI engine 122 may use imaging extraction techniques, such as optical character recognition (OCR) and/or use a machine learning model trained to identify and extract information from images. OCR refers to electronic conversion of an image of printed text into machine-encoded text. As another example, pattern recognition and/or computer vision may also be used to process images. Computer vision may involve image understanding by processing symbolic information from image data using models constructed with the aid of geometry, physics, statistics, and/or learning theory. Pattern recognition may refer to electronic discovery of regularities in data through the use of computer algorithms and with the use of these regularities to take actions such as classifying the data into different categories and/or determining what the symbols represent in the image (e.g., words, sentences, names, numbers, identifiers, etc.). Cognitive AI engine 122 may also use natural language understanding (NLU) techniques to process unstructured data using text analytics to extract entities, relationships, keywords, semantic roles, and so forth. Furthermore, cognitive AI engine 122 may use sentiment analysis to identify, extract, and quantify subjective information from medical information (such as subjective information from notes taken by a medical provider) associated with a participant. Cognitive AI engine 122 may use the same technologies to synthesize data from various information sources, while weighing context and conflicting evidence, to determine a likelihood that a participant has contracted the infectious medical condition.

Cognitive AI engine 122 can be configured to train the ML models based on medical information associated with participants. Additionally, cognitive AI engine 122 can be configured to update the ML models based on medical information. For example, cognitive AI engine 122 may maintain the ML models by continuously retraining the ML models based on medical information. For example, the medical provider may be a physician that performed a medical test on the participant and the medical information may include the type of medical test and the result of the medical test, among other information. In some embodiments, the medical information may include information pertaining to a medical test performed for the patient, a medical metric pertaining to the patient, a result of the medical test performed for the patient, a license of the medical personnel, a degree of the medical personnel, a timestamp of the medical information, or some combination thereof. Further, cognitive AI engine 122 may apply the medical information to the ML models and receive an indication, from the ML models, of a likelihood (e.g., a likelihood score that the second participant contracted the infectious medical condition) that a participant has contracted the infectious medical condition.

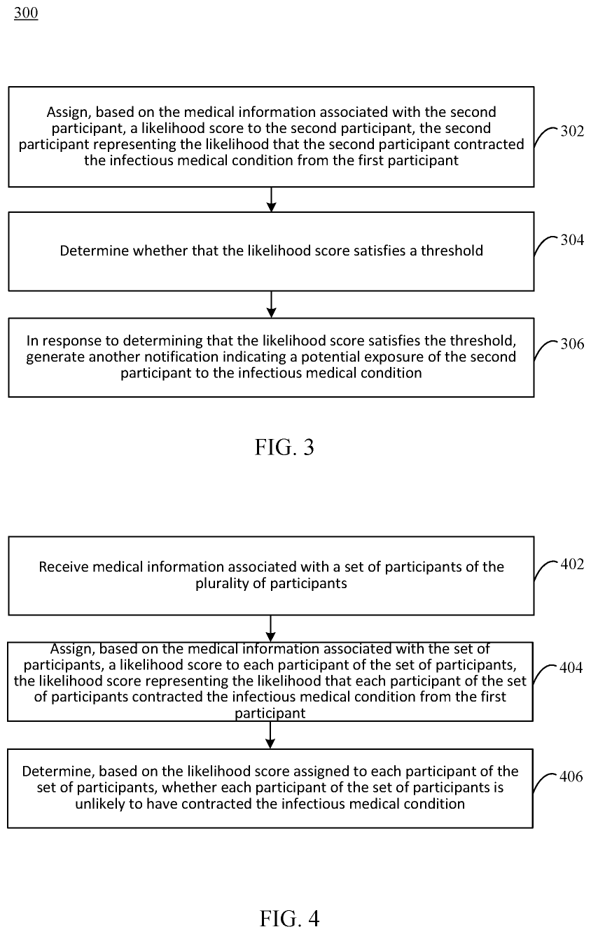

FIG. 3 will now be described. FIG. 3 generally illustrates a method 300 for assigning, based on medical information associated with a participant, a likelihood score to the participant and based on the likelihood score, generating a notification indicating a potential exposure of the participant to an infectious medical condition. As shown in FIG. 3, method 300 starts at step 302. At step 302, based on the medical information associated with the second participant, a likelihood score is assigned to the second participant, where the likelihood score represents the likelihood that the second participant contracted the infectious medical condition from the first participant. For example, with continued reference to FIG. 1, integrated contact tracing platform 104 may assign, based on the medical information associated with participant 120, a likelihood score (e.g., a percentage, a number, or any other desired score indicator) to the second participant. In an embodiment, the medical information may indicate that the medical test was administered to participant 120 and the notes of the healthcare provider may confirm a positive result of the medical test reported by a laboratory. In such a case, integrated contact tracing platform 104 may assign a high likelihood score that participant 120 has contracted the infectious medical condition. In an embodiment, notes from a doctor may reveal symptoms participant 120 may be experiencing. If the symptoms do not match symptoms associated with the infectious medical condition, then integrated contact tracing platform 104 may assign a lower likelihood score that participant 120 has contracted the infectious medical condition.

At step 304, it is determined whether the likelihood score satisfies a threshold representing a probability of contracting the infectious medical condition. For example, with continued reference to FIG. 1, integrated contact tracing platform 104 may determine whether the likelihood score satisfies the threshold (e.g., above 70% probability of contracting the infectious medical condition) by comparing the likelihood scores to the threshold.

At step 306, in response to determining that the likelihood score satisfies the threshold, another notification is generated, where the other notification indicates a potential exposure of the second participant to the infectious medical condition. For example, with continued reference to FIG. 1, integrated contact tracing platform 104 may generate a notification (e.g., text message, voice message, etc.) indicating a potential exposure of participant 120 to the infectious medical condition. For example, integrated contact tracing platform 104 may generate a notification for participant 120 and provide the notification to computing device 112 to be displayed on user interface 116.

FIG. 4 will now be described. FIG. 4 shows a method 400 for assigning, based on medical information associated with each participant of a set of participants, a likelihood score to each participant of the set of participants. As shown in FIG. 4, method 400 starts at step 402. At step 402, medical information associated with a set of participants of the plurality of participants is received. For example, with continued reference to FIG. 1, integrated contact tracing platform 104 may receive medical information associated with a set of participants of the plurality of participants.

At step 404, based on the medical information associated with the set of participants, a likelihood score is assigned to each participant of the set of participants, where the likelihood score represents the likelihood that each participant of the set of participants contracted the infectious medical condition from the first participant. For example, with continued reference to FIG. 1, integrated contact tracing platform 104 may assign, based on the medical information associated with the set of participants, a likelihood score to each participant of the set of participants, the likelihood score representing the likelihood that each participant of the set of participants contracted the infectious medical condition from participant 118.

At step 406, based on the likelihood score assigned to each participant of the set of participants, it is determined whether each participant of the set of participants is unlikely to have contracted the infectious medical condition. For example, with continued reference to FIG. 1, integrated contact tracing platform 104 may determine, based on the likelihood score assigned to each participant of the set of participants, whether each participant of the set of participants is unlikely to have contracted the infectious medical condition. For example, if the likelihood score for any of the participants of the plurality of participants is below a threshold (e.g., below 70%), integrated contact tracing platform 104 may determine that such participants of the set of participants are unlikely to have contracted the infectious medical condition.

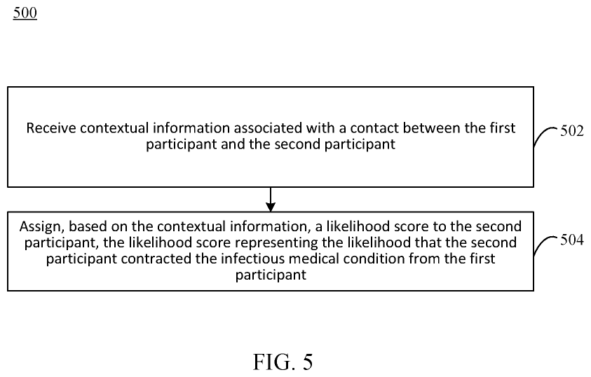

FIG. 5 will now be described. FIG. 5 shows a method 500 for assigning, based on the contextual information, a likelihood score to the second participant. As shown in FIG. 5, method 500 starts at step 502. At step 502, contextual information is received, where the contextual information is associated with a contact between the first participant and the second participant. For example, with continued reference to FIG. 1, integrated contact tracing platform 104 may receive contextual information associated with a contact between participant 118 and participant 120. The contextual information may include one or more of the following: a distance between the first participant and second participant, a set of attributes pertaining to a place of the contact, a number of people involved in the contact, a purpose or nature of the contact, any other desired contextual information, or combination thereof. For example, participant 118 and participant 120 may provide contextual information associated with a contact that they may have with other people via user interfaces 114 and 116, respectively. In an embodiment, integrated contact tracing platform 104 may prompt participant 118 and participant 120 to answer questions concerning the contact via user interface 114 and 116, respectively. The questions may pertain to how the infectious medical condition is transmitted between people. For example, the questions for participant 118 may include if participant 116 was within a certain distance from another person, including participant 120; exhibited any symptoms, such as coughing or sneezing, at a certain location; physically contacted another person, including participant 120, and the like. The questions for participant 120 may include if participant 120 was within a certain distance from participant 118; if participants 118 exhibited any symptoms, such as coughing or sneezing, in the presence of participant 120; physically contacted participant 118, and the like.

At step 504, based on the contextual information, a likelihood score is assigned to the second participant, where the likelihood score represents the likelihood that the second participant contracted the infectious medical condition from the first participant. For example, with continued reference to FIG. 1, integrated contact tracing platform 104 may assign, based on the contextual information, a likelihood score to participant 120. For example, if the infectious medical condition is spread by pathogens in the air, the contextual information such as the contact occurring indoors or within a confined space, the participants 118 and 120 may be determined to have had physical contact with each other. If contact is determined and the contact lasted an extended period of time (e.g., more than an hour), then integrated contact tracing platform 104 may assign a high likelihood score to participant 120. Other factors can be used separately or in combination with each other to determine the likelihood score and are not limited by the examples provided in this disclosure.

FIGS. 2-5 are not intended to be limiting: the methods 200, 300, 400, and 500 can include more or fewer steps and/or processes than those illustrated in FIGS. 2-5. Further, the order of the steps of the methods 200, 300, 400, and 500 is not intended to be limiting; the steps can be arranged in any suitable order.

Embodiments described herein provide several technical benefits. One such technical benefit provided is saving computing resources (e.g., processing, network, memory, etc.). For example, determining which participants, who came into contact with an infected participant, are unlikely to have contracted the infectious medical condition from the infected person (e.g., because of immunity, a negative medical test result, a lack of symptoms, etc.) saves computing resources. This reduces the number of participants who need to be notified and eliminates identifying additional participants who came into contact with a participant unlikely to have contracted the infectious medical condition. Also, the user interface (e.g., user interface 114 and 116) enables a user to provide contextual information of a contact with another participant, thereby providing an improved user interface that may increase the user's experience using the client computing device (e.g., client computing device 110 and 112) and/or integrated contact tracing platform 104.

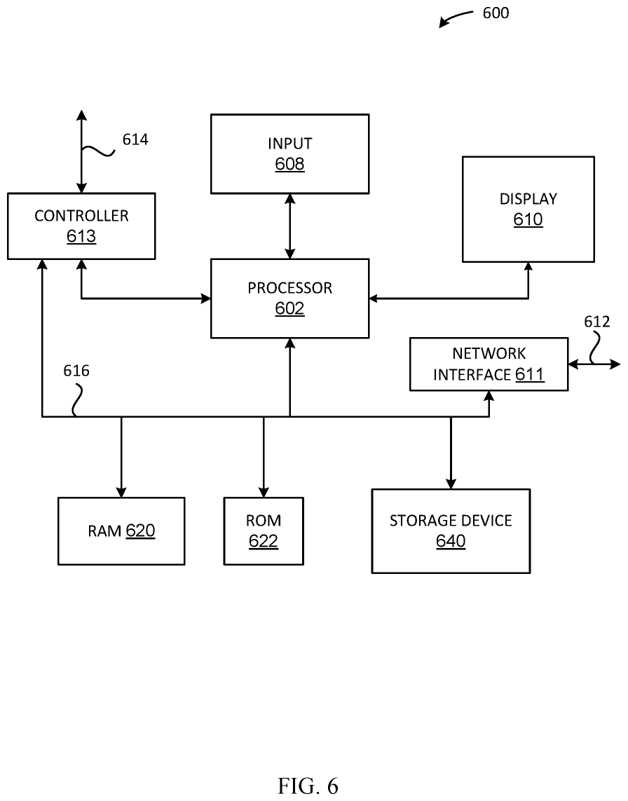

FIG. 6 generally illustrates a high-level component diagram of a computing device 600 that can be used to implement the various components described herein, according to some embodiments. In particular, the diagram illustrates various components that can be included in client computing devices 110 and 112 and sources 106A, 106B, 106C . . . 106N as illustrated in FIG. 1. As shown in FIG. 6, computing device 600 can include a processor 602 that represents a microprocessor or controller for controlling the overall operation of computing device 600. Computing device 600 can also include a user input device 608 that allows a user of computing device 600 to interact with computing device 600. For example, user input device 608 can take a variety of forms, such as a button, keypad, dial, touch screen, audio input interface, visual/image capture input interface, input in the form of sensor data, and so on. Still further, computing device 600 can include a display 610 that can be controlled by processor 602 to display information to the user. A data bus 616 can facilitate data transfer between at least a storage device 640, processor 602, and a controller 613. Controller 613 can be used to interface with and control different equipment through an equipment control bus 606. Computing device 600 can also include a network/bus interface 611 that couples to a data link 612. In the case of a wireless connection, network/bus interface 611 can include a wireless transceiver.

As noted above, computing device 600 also includes storage device 640, which can comprise a single disk or a collection of disks (e.g., hard drives), and includes a storage management module that manages one or more partitions within storage device 640. In some embodiments, storage device 640 can include flash memory, semiconductor (solid-state) memory or the like. Computing device 600 can also include a Random-Access Memory (RAM) 620 and a Read-Only Memory (ROM) 622. ROM 622 can store programs, utilities or processes to be executed in a non-volatile manner. RAM 620 can provide volatile data storage, and can store instructions related to the operation of processes and applications executing on the computing device 600.

FIG. 6 is not intended to be limiting: computing device 600 may include more or fewer components than those illustrated in FIG. 6.

The various aspects, embodiments, implementations or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data which can thereafter be read by a computer system. Examples of the computer readable medium include read-only memory, random-access memory, CD-ROMs, DVDs, magnetic tape, hard disk drives, solid-state drives, and optical data storage devices. The computer readable medium can also be distributed over network-coupled computer systems so that the computer readable code is stored and executed in a distributed fashion.

Consistent with the above disclosure, the examples of systems and method enumerated in the following clauses are specifically contemplated and are intended as a non-limiting set of examples.

In an embodiment, a computer-implemented method for a contact tracing protocol performed by an integrated contact tracing platform, the method comprises: receiving location information associated with a first participant in the contact tracing protocol; receiving medical information associated with the first participant, the medical information indicating the first participant has contracted an infectious medical condition; identifying, based on the location information, a plurality of participants in the contact tracing protocol who came into contact with the first participant during a contagious period associated with the infectious medical condition; receiving medical information associated with a second participant of the plurality of participants, the medical information related to the infectious medical condition; determining, based on the medical information associated with the second participant, a likelihood that the second participant has contracted the infectious medical condition from the first participant; and generating a notification for each participant of the plurality of participants indicating a potential exposure to the infectious medical condition.

In a foregoing embodiment, the medical information includes an indication of a positive result of a medical test administered to the first participant, wherein the medical test is configured to detect or aid in diagnosis of the infectious medical condition.

In a foregoing embodiment, the method further comprises: identifying, based on the location information, another plurality of participants in the contact tracing protocol who came into contact with an environment or a surface that the first participant may have contaminated with the infectious medical condition during the contagious period associated with the infectious medical condition.

In a foregoing embodiment, determining the likelihood that the second participant has contracted the infectious medical condition comprises: assigning, based on the medical information associated with the second participant, a likelihood score to the second participant, the likelihood score representing the likelihood that the second participant contracted the infectious medical condition from the first participant; and the method comprising: determining whether that the likelihood score satisfies a threshold; and in response to determining that the likelihood score satisfies the threshold, generating a second notification indicating a potential exposure of the second participant to the infectious medical condition.

In a foregoing embodiment, the medical information associated with the second participant includes an indication of a negative result of a medical test administered to the second participant, wherein the medical test is administered after a contact between the first participant and the second participant and the medical test is configured to detect or aid in diagnosis of the infectious medical condition.

In a foregoing embodiment, the medical information associated with the second participant includes indications of symptoms associated with the infectious medical condition experienced by the second participant.

In a foregoing embodiment, the method further comprises: receiving medical information associated with a set of participants of the plurality of participants; assigning, based on the medical information associated with the set of participants, a likelihood score to each participant of the set of participants, the likelihood score representing the likelihood that each participant of the set of participants contracted the infectious medical condition from the first participant; and determining, based on the likelihood score assigned to each participant of the set of participants, whether each participant of the set of participants is likely to have contracted the infectious medical condition.

In a foregoing embodiment, the method comprises: receiving contextual information associated with a contact between the first participant and the second participant; and assigning, based on the contextual information, a likelihood score to the second participant, the likelihood score representing the likelihood that the second participant contracted the infectious medical condition from the first participant.

In a foregoing embodiment, the contextual information includes one or more of the following: a distance between the first participant and second participant; a set of attributes pertaining to a place of the contact; a number of people involved in the contact; and a purpose or nature of the contact.

In an embodiment, a system, comprises: a memory storing instructions that implement an application for reconciling electronic health records of a patient; and a processing device communicatively coupled to the memory, the processing device capable of executing the application to: receive location information associated with a first participant in the contact tracing protocol; receive medical information associated with the first participant, the medical information indicating the first participant has contracted an infectious medical condition; identify, based on the location information, a plurality of participants in the contact tracing protocol who came into contact with the first participant during a contagious period associated with the infectious medical condition; receive medical information associated with a second participant of the plurality of participants, the medical information related to the infectious medical condition; determine, based on the medical information associated with the second participant, a likelihood that the second participant has contracted the infectious medical condition from the first participant; and generate a notification for each participant of the plurality of participants indicating a potential exposure to the infectious medical condition.

In a foregoing embodiment, the medical information includes an indication of a positive result of a medical test administered to the first participant, wherein the medical test is configured to detect or aid in diagnosis of the infectious medical condition.

In a foregoing embodiment, the processing device is further capable of executing the application to: identify, based on the location information, a plurality of participants in the contact tracing protocol who came into contact with an environment or a surface that the first participant may have contaminated with the infectious medical condition during the contagious period associated with the infectious medical condition.

In a foregoing embodiment, the processing device is further capable of executing the application to: assign, based on the medical information associated with the second participant, a likelihood score to the second participant, the second participant representing the likelihood that the second participant contracted the infectious medical condition from the first participant; determine whether that the likelihood score satisfies a threshold; and in response to determining that the likelihood score satisfies the threshold, generate a second notification indicating a potential exposure of the second participant to the infectious medical condition.

In a foregoing embodiment, generating the notification for each participant of the plurality of participants includes excluding the second participant in response to determining that the likelihood score does not satisfy the threshold.

In a foregoing embodiment, the medical information associated with the second participant includes an indication of a negative result of a medical test administered to the second participant, wherein the medical test is administered after a contact between the first participant and the second participant and the medical test is configured to detect or aid in diagnosis of the infectious medical condition.

In a foregoing embodiment, the medical information associated with the second participant includes indications of symptoms associated with the infectious medical condition experienced by the second participant.

In a foregoing embodiment, the processing device is further capable of executing the application to: receive medical information associated with a set of participants of the plurality of participants; assign, based on the medical information associated with the set of participants, a likelihood score to each participant of the set of participants, the likelihood score representing the likelihood that each participant of the set of participants contracted the infectious medical condition from the first participant; and determine, based on the likelihood score assigned to each participant of the set of participants, whether each participant of the set of participants is likely to have contracted the infectious medical condition.

In a foregoing embodiment, the processing device is further capable of executing the application to: receive contextual information associated with a contact between the first participant and the second participant; and assign, based on the contextual information, a likelihood score to the second participant, the likelihood score representing the likelihood that the second participant contracted the infectious medical condition from the first participant.

In an embodiment, a tangible, non-transitory computer-readable medium storing instructions that, when executed, cause a processing device to: receive location information associated with a first participant in the contact tracing protocol; receive medical information associated with the first participant, the medical information indicating the first participant has contracted an infectious medical condition; identify, based on the location information, a plurality of participants in the contact tracing protocol who came into contact with the first participant during a contagious period associated with the infectious medical condition; receive medical information associated with a second participant of the plurality of participants, the medical information related to the infectious medical condition; determine, based on the medical information associated with the second participant, a likelihood that the second participant has contracted the infectious medical condition from the first participant; and generate a notification for each participant of the plurality of participants indicating a potential exposure to the infectious medical condition.

In a foregoing embodiment, the medical information includes an indication of a positive result of a medical test administered to the first participant, wherein the medical test is configured to detect or aid in diagnosis of the infectious medical condition.

In the embodiment of the foregoing system, the processing device is further capable of executing the application to: determine a portion of the list of health providers to be provided the notification of the inconsistency or the contraindication based on a categorical grouping of the inconsistency or the contraindication.

The foregoing description, for purposes of explanation, used specific nomenclature to provide a thorough understanding of the described embodiments. However, it should be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It should be apparent to one of ordinary skill in the art that many modifications and variations are possible in view of the above teachings.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

1. A computer-implemented method for a contact tracing protocol performed by an integrated contact tracing platform, the method comprising:
generating, with a cognitive artificial intelligence engine, one or more machine learning models trained to determine a likelihood that a person has contacted an infectious medical condition during a contact with an infected participant, wherein the cognitive artificial intelligence engine is configured to train the one or more machine learning models with training data comprising medical information related to medical tests on participants including at least one selected from the group consisting of types of the medical tests, results of the medical tests, licenses of medical personnel administering the medical tests, degrees of the medical personnel administering the medical tests, and timestamps of the medical tests;
receiving location information associated with a first participant in the contact tracing protocol;
receiving medical information associated with the first participant, the medical information indicating the first participant has contracted the infectious medical condition;
identifying, based on the location information, a plurality of participants in the contact tracing protocol who came into contact with the first participant during a contagious period associated with the infectious medical condition;
receiving medical information associated with a second participant of the plurality of participants, the medical information related to the infectious medical condition;
determining, using the one or more machine learning models and based on the medical information associated with the second participant, a likelihood that the second participant has contracted the infectious medical condition from the first participant; and
generating a notification for each participant of the plurality of participants indicating a potential exposure to the infectious medical condition.

2. The method of claim 1, wherein the medical test is a first medical test, wherein the medical information associated with the first participant includes an indication of a positive result of a second medical test administered to the first participant, and wherein the second medical test is configured to detect or aid in diagnosis of the infectious medical condition.

3. The method of claim 1, the method further comprising:
identifying, based on the location information, another plurality of participants in the contact tracing protocol who came into contact with an environment or a surface that the first participant may have contaminated with the infectious medical condition during the contagious period associated with the infectious medical condition.

4. The method of claim 1, wherein determining the likelihood that the second participant has contracted the infectious medical condition comprises:
assigning, based on the medical information associated with the second participant, a likelihood score to the second participant, the likelihood score representing the likelihood that the second participant contracted the infectious medical condition from the first participant; and the method comprising:
determining whether that the likelihood score satisfies a threshold; and
in response to determining that the likelihood score satisfies the threshold, generating a second notification indicating a potential exposure of the second participant to the infectious medical condition.

5. (canceled)

6. The method of claim 1, wherein the medical information associated with the second participant includes indications of symptoms associated with the infectious medical condition experienced by the second participant.

7. The method of claim 1, the method further comprising:
receiving medical information associated with a set of participants of the plurality of participants;
assigning, based on the medical information associated with the set of participants, a likelihood score to each participant of the set of participants, the likelihood score representing the likelihood that each participant of the set of participants contracted the infectious medical condition from the first participant; and
determining, based on the likelihood score assigned to each participant of the set of participants, whether each participant of the set of participants is likely to have contracted the infectious medical condition.

8. The method of claim 1, the method comprising:
receiving contextual information associated with a contact between the first participant and the second participant; and
assigning, based on the contextual information, a likelihood score to the second participant, the likelihood score representing the likelihood that the second participant contracted the infectious medical condition from the first participant.

9. The method of claim 8, wherein the contextual information includes one or more of the following: a distance between the first participant and the second-participant; a set of attributes pertaining to a place of the contact; a number of people involved in the contact; and a purpose or nature of the contact.

10. A system, comprising:
a memory storing instructions that implement an application for reconciling electronic health records of a patient; and
a processing device communicatively coupled to the memory, the processing device capable of executing the application to:
generate, with a cognitive artificial intelligence engine, one or more machine learning models trained to determine a likelihood that a person has contacted an infectious medical condition during a contact with an infected participant, wherein the cognitive artificial intelligence engine is configured to train the one or more machine learning models with training data comprising medical information related to medical tests on participants including at least one selected from the group consisting of types of the medical tests, results of the medical tests, licenses of medical personnel administering the medical tests, degrees of the medical personnel administering the medical tests, and timestamps of the medical tests;
receive location information associated with a first participant in a contact tracing protocol;
receive medical information associated with the first participant, the medical information indicating the first participant has contracted the infectious medical condition;
identify, based on the location information, a plurality of participants in the contact tracing protocol who came into contact with the first participant during a contagious period associated with the infectious medical condition;
receive medical information associated with a second participant of the plurality of participants, the medical information related to the infectious medical condition;
determine, using the one or more machine learning models and based on the medical information associated with the second participant, a likelihood that the second participant has contracted the infectious medical condition from the first participant; and
generate a notification for each participant of the plurality of participants indicating a potential exposure to the infectious medical condition.

11. The system of claim 10, wherein the medical test is a first medical test, wherein the medical information associated with the first participant includes an indication of a positive result of a second medical test administered to the first participant, and wherein the second medical test is configured to detect or aid in diagnosis of the infectious medical condition.

12. The system of claim 10, wherein the processing device is further capable of executing the application to:
identify, based on the location information, a plurality of participants in the contact tracing protocol who came into contact with an environment or a surface that the first participant may have contaminated with the infectious medical condition during the contagious period associated with the infectious medical condition.

13. The system of claim 10, wherein the processing device is further capable of executing the application to:
assign, based on the medical information associated with the second participant, a likelihood score to the second participant, the second participant representing the likelihood that the second participant contracted the infectious medical condition from the first participant;
determine whether that the likelihood score satisfies a threshold; and
in response to determining that the likelihood score satisfies the threshold, generate a second notification indicating a potential exposure of the second participant to the infectious medical condition.

14. The system of claim 13, wherein generating the notification for each participant of the plurality of participants includes excluding the second participant in response to determining that the likelihood score does not satisfy the threshold.

15. (canceled)

16. The system of claim 11, wherein the medical information associated with the second participant includes indications of symptoms associated with the infectious medical condition experienced by the second participant.

17. The system of claim 11, wherein the processing device is further capable of executing the application to:
receive medical information associated with a set of participants of the plurality of participants;
assign, based on the medical information associated with the set of participants, a likelihood score to each participant of the set of participants, the likelihood score representing the likelihood that each participant of the set of participants contracted the infectious medical condition from the first participant; and
determine, based on the likelihood score assigned to each participant of the set of participants, whether each participant of the set of participants is likely to have contracted the infectious medical condition.

18. The system of claim 11, wherein the processing device is further capable of executing the application to:
receive contextual information associated with a contact between the first participant and the second participant; and
assign, based on the contextual information, a likelihood score to the second participant, the likelihood score representing the likelihood that the second participant contracted the infectious medical condition from the first participant.

19. A tangible, non-transitory computer-readable medium storing instructions that, when executed, cause a processing device to:
generate, with a cognitive artificial intelligence engine, one or more machine learning models trained to determine a likelihood that a person has contacted an infectious medical condition during a contact with an infected participant, wherein the cognitive artificial intelligence engine is configured to train the one or more machine learning models with training data comprising medical information related to medical tests on participants including at least one selected from the group consisting of types of the medical tests, results of the medical tests, licenses of medical personnel administering the medical tests, degrees of the medical personnel administering the medical tests, and timestamps of the medical tests;
receive location information associated with a first participant in a contact tracing protocol;
receive medical information associated with the first participant, the medical information indicating the first participant has contracted the infectious medical condition;
identify, based on the location information, a plurality of participants in the contact tracing protocol who came into contact with the first participant during a contagious period associated with the infectious medical condition;
receive medical information associated with a second participant of the plurality of participants, the medical information related to the infectious medical condition;
determine, using the one or more machine learning models and based on the medical information associated with the second participant, a likelihood that the second participant has contracted the infectious medical condition from the first participant; and
generate a notification for each participant of the plurality of participants indicating a potential exposure to the infectious medical condition.

20. The tangible, non-transitory computer-readable medium of claim 19, wherein the medical test is a first medical test, wherein the medical information associated with the first participant includes an indication of a positive result of a second medical test administered to the first participant, and wherein the second medical test is configured to detect or aid in diagnosis of the infectious medical condition.
